(19) United States
(12) Patent Application Publication
(10) Pub. No.: US 2026/0115106 A1
(43) Pub. Date: Apr. 30, 2026

(54) ANTIMICROBIAL DENTAL MATERIAL COMPOSITIONS FOR LUBRICATION, CLEANSING AND REMOVAL OF THE SMEAR LAYER FROM THE ROOT CANAL

(71) Applicant: INTER-MED, INC., Racine, WI (US)

(72) Inventors: Alexander D. Johnson, Racine, WI (US); Brett Arand, Milwaukee, WI (US); Ashir Mahendra, Racine, WI (US); Allen Ali Nasseh, Racine, WI (US)

(21) Appl. No.: 19/003,498

(22) Filed: Dec. 27, 2024

Related U.S. Application Data
(60) Provisional application No. 63/615,217, filed on Dec. 27, 2023.

(51) Int. Cl. A61K 6/52; A61K 6/60; A61K 6/69

(52) U.S. Cl. CPC A61K 6/52; A61K 6/60; A61K 6/69

(57) ABSTRACT

Endodontic lubricant compositions are disclosed for use in RCT in vivo improve endodontic file longevity. Methods of using the compositions, kits containing the compositions, and manufacturing processes to create the compositions are also disclosed.

### CROSS-REFERENCE TO RELATED APPLICATIONS

This application claims the benefit of U.S. Provisional Application No. 63/615,217, filed Dec. 27, 2023, the entire contents of which are incorporated herein by reference.

### BACKGROUND OF INVENTION

Human teeth are living structures comprised of both hard and soft tissues. The hard tissue consists of enamel (the hard exterior surface tissue of teeth), dentin (immediately below enamel and helps support enamel), and cementum (a specialized hard tissue that covers the root of a tooth). The dental pulp is generally referred to as soft tissue and primarily resides within the pulp chamber. Pulp tissue consists of many types of cells and connective tissue but also includes blood vessels and nerves that enter the tooth from the apical foramen which is a hole at the apex of a root canal. Generally, a tooth's root canal system refers to the naturally occurring anatomical spaces within the root(s) of a tooth including the pulp chamber.

The root canal system is complex and can become infected in several ways. Commonly this occurs if dental caries are not treated before pulp and root canal infiltration by bacteria. Once a root canal system is infected, many treatment methods can be used depending upon the condition of the patient and approach of the practitioner. Typically, an endodontic procedure called root canal therapy (RCT) is performed to cleanse the root canal system and remove infectious agents.

RCT usually involves three main steps. The first step is called instrumentation and entails opening the tooth and widening the root canal system utilizing a series of semi-rigid metal endodontic files, often times with endodontic irrigants (e.g. sodium hypochlorite) to reduce the debris level created by the endodontic files. This step removes some organic and inorganic material, but its primary goal is to enlarge the root canal(s) to allow for the introduction of small cannula used during the second step, called irrigation. Irrigation entails flushing of the root canal system using small cannula (called irrigation needles) attached to syringes of various aqueous-based endodontic irrigants, including sodium hypochlorite. The goal of the irrigation step is to debride the root canal system and solubilize any remaining debris and smear layer introduced during instrumentation. The third and final step is called obturation, in which the root canal system is filled with various materials. After the endodontic procedure is finalized, a standard dental restorative procedure (i.e., composite filling or crown, among others) is used to complete the structure and aesthetics of the tooth.

Although a liquid irrigant is typically used for smear layer removal, the use of a gel based solution may be beneficial to help suspend and lift debris from the canal during the instrumentation step of RCT. Furthermore, the thicker consistency of gels allows them to cling to endodontic files, and specifically within the files' flute spaces, and be placed directly into the patient's tooth, which allows the dental professional increased application flexibility. Gel solutions may also help lubricate the endodontic file and canal to improve dentin cutting ability, prevent endodontic file binding, and prolong endodontic file life.

Focusing on endodontic creams and lubricants, U.S. Pat. App. No. US20170348199A1 describes a composition and method for use in a dental treatment such as a root canal where the composition is a mixture of a lubricant, disinfectant, an anesthetic, and a chelating agent. This composition is made up of cetyl alcohol, urea peroxide, ethylenediaminetetraacetic (EDTA), and epinephrine in a water-soluble base.

The commercially available product RC Prep (Premier Dental Products, Plymouth Meeting, PA, USA), is the market leader for an endodontic gel material, and its composition is a mixture of glycol, urea peroxide, EDTA in a water-soluble base. Although RC Prep has been successful commercially, its limitations include: 1) the material is too thick that it can't be easily applied into the middle and apical thirds of the root canal where root canal shaping is most important, 2) despite use of the material, endodontic file flutes still get plugged with smear layer and dentin debris and have to be removed from the tooth and wiped clean, which leads to unproductive down time for the dental professional, 3) the material is cytotoxic, 4) prolonged dentin exposure to high concentrations of EDTA may result in excessive dentin demineralization and decreased strength, and 5) the material does not contain additional debriding or antimicrobial agents to improve cleaning.

For cleaning compositions containing an antimicrobial agent, U.S. Pat. No. 11,297,839 describes an antimicrobial composition consisting of iodine (I₂), iodate (IO³⁻), and an acid, with defined specifications related to the molar ratio of iodine to iodate and the acid for use as a pH modifier.

Irrespective of prior developments, there exists a need for an improved endodontic material that is more flowable to easily reach all areas of root canal complexity, keeps endodontic file flutes clear of debris to limit procedural downtime, is more biocompatible and dentin friendly, and provides additional debriding and antimicrobial properties.

### BRIEF DESCRIPTION OF DRAWINGS

FIG. 1 consists of a series of laser microscope images that show satisfactory smear layer removal within the root canal using the disclosed lubricant composition.

### DESCRIPTION OF THE INVENTION

The following paragraphs define in more detail the embodiments described herein. The following embodiments are not meant to limit the invention or narrow the scope thereof, as it will be readily apparent to one of ordinary skill in the art that suitable modifications and adaptations may be made without departing from the scope of the invention, embodiments, or specific aspects described herein.

The present disclosure provides an improved lubricant composition for use in endodontic treatments, particularly for use in RCT in vivo. The disclosed composition exhibits significant advantages compared to products presently on the market including: 1) improved endodontic file longevity by keeping the file lubricated and flutes clean during instrumentation, 2) enhanced antimicrobial properties, 3) improved debris clearing and debridement, 4) improved biocompatibility (i.e., less cytotoxic) compared to other endodontic materials, 5) ability to extrude through a smaller tip allowing for better access into the canal and its restrictions, and 6) PH balanced to be effective, safe, and dentin friendly (i.e., dentin is not excessively eroded). In further embodiments, methods of use of the disclosed compositiosns are described in detail. In yet another aspect, a product by process to create the lubricant composition is disclosed. In an even further aspect, kits containing the composition are disclosed.

Depending on the clinical use, the lubricant composition may be a liquid or a gel like material. The composition is generally comprised of at least one chelating agent, at least one lubricant, at least one surfactant, and at least one solvent. Optionally, the composition may additionally comprise at least one humectant, at least one debriding agent, at least one rheology modifier, at least one effervescent agent, or at least one antimicrobial agent. In certain embodiments, the debriding agent and antimicrobial agent may be the same chemical or compound. In certain embodiments, the chelating agent and debriding agent may be the same chemical or compound. In certain embodiments, the lubricant and surfactant may be the same chemical or compound. In certain embodiments, the lubricant and humectant may be the same chemical or compound. To mitigate any potential confusion, the “same chemical or compound” refers to the same active ingredient being used; therefore, different salts or hydrates of the same active ingredient would still be considered the same chemical or compound.

The chelating agent is preferably EDTA, citric acid, 2-phosphonobutane-1,2,4,-tricarboxylic acid (PBTC), etidronic acid (HEDP), polyacrylic acid, or combinations thereof. The molecular weight of polyacrylic acid is preferably between 1000 g/mol-190,000 g/mol, and more preferably between 1000 g/mol-10,000 g/mol. The chelating agent is preferably at a weight/weight concentration (% w/w) between 1-50%, and more preferably between 5-20%, and most preferably between 7.5-15%.

The lubricant composition can exhibit different pHs depending on the composition and clinical utility. In certain embodiments, the lubricant composition has a neutral pH preferably between 6.0-8.0 and most preferably between 7.0 and 8.0. This pH range promotes optimal chelation performance for certain chelating agents, such as EDTA, compared to a more acidic formulation. Furthermore, a neutral pH provides a more dentin friendly method for smear layer removal along with improved biocompatibility. Alternatively, other compositions utilizing different chelating agents, such as citric acid, perform better in an acidic environment along with optional antimicrobial additives such as elemental iodine (I₂). I₂ may also be considered a debriding agent. In these embodiments, the lubricant composition has an acidic pH preferably between 2.0-7.0, more preferably between 2.0-6.0, and most preferably between 2.0 and 4.5. Here, a balanced acidic pH is needed to ensure chelation and antimicrobial efficacy without excessive dentin demineralization/erosion and toxicity. In other words, the pH can't be too acidic as that may cause excessive dentin erosion and cell cytotoxicity, but it needs to be somewhat acidic for chelation and antimicrobial efficacy.

The lubricant is preferably propylene glycol, glycerin, polyethylene glycol, boric acid, sodium oleate, sodium benzoate, sodium lauryl sulphate, sodium acetate, amongst others. The lubricant is preferably at a weight/weight concentration (% w/w) between 1-50%, more preferably between 1-20%, and most preferably between 2.5-10%. In certain embodiments, the lubricant is hydrophilic although hydrophobic lubricants may be utilized with a water miscible solvent such as ethanol, isopropyl alcohol, amongst others.

The lubricant composition may also include surfactants to enhance wetting, solubilization (solubility, cleaning and debris removal), debris suspension, debridement, and emulsification. Examples of suitable surfactants (also referred to as wetting agents) include alkyldiphenyloxide disulfonates, alkyl aryl sulfonates, sodium dodecylbenzene sulfonate, alkyl sulfates, alcohol ethoxylates, polyoxyethylene glycol octylphenol ethers, polyoxyethylene glycol alkylphenol ethers, polyoxyethylene glycol sorbitan alkyl esters, sorbitan alkyl esters, copolymers of polyethylene glycol and/or propylene glycol, poloxamers, sodium sterates, sodium lauryl ether sulfates, linear alkylbenzene sulfonates, amine oxides, betaines, sodium lauryl sulfate, and disulfonates, fluorosurfactants, among others. In some embodiments, the surfactants are nonionic, anionic or zwitterionic, although certain cationic surfactants can be employed. The surfactant is preferably at a weight/weight concentration (% w/w) between 0.1-20%, more preferably between 0.5-10%, and most preferably between 1-10%. In certain embodiments, the surface tension of the lubricant composition is preferably less than 100 dynes/cm, more preferably less than 60 dynes/cm, and most preferably less than 40 dynes/cm.

Depending on clinical use, a debriding agent or antimicrobial agent may be additionally added to the lubricant composition. Suitable debriding and antimicrobial agents include elemental iodine (I₂), hypochlorous acid, chlorhexidine, amongst others. In certain embodiments, the lubricant composition preferably provides at least a log 3 antimicrobial impact within one minute of use, but more preferably provides at least a log 7 antimicrobial impact within one minute of use. In preferred embodiments, elemental iodine (I₂) is added to the lubricant composition (as I₂ simultaneously exhibits debriding and antimicrobial properties) at a preferred concentration between 10 parts per million (PPM)-10,000 PPM, a more preferred concentration between 100 PPM-5,000 PPM, and a most preferred concentration between 300 PPM-3,000 PPM. In such a composition, an acidic pH less than 6 is preferred for 12 stability and efficacy. Furthermore, the concentration of I₂ shall be selected such that brown staining of the dentin does not occur during or after use which would not be aesthetically pleasing in the mouth (although sodium hypochlorite is subsequently used in endodontic procedures which bleaches any I₂ staining). In other words, in certain embodiments the lubricant composition is non-staining. When I₂ is utilized in the lubricant composition, PBTC cannot be used as the chelating agent and surfactants cannot comprise fluorosurfactants or amine oxides. Additionally, optional stabilizers may be added to the lubricant composition comprising I₂ to help promote I₂ aqueous stability and concentration; however, unlike the prior art, the use of iodate (IO³⁻) is not necessary for stabilizing the disclosed compositions. Suitable stabilizers include potassium iodide (KI), sodium chloride (NaCl), iodide salts, chloride salts, ethanol, alcohols, amongst others. I₂ is preferred compared to other debriding agents or antimicrobial agents due to its biocompatibility and lower toxicity. Additionally in certain embodiments, an I₂ based lubricant composition may be appreciably odorless or less noxious.

In some embodiments the lubricant composition additionally comprises a rheology modifier that may also optionally serve as a thickener to improve the ability to control the flow of the lubricant during application. A preferred example is silicon dioxides (for example, fumed silica), preferably at a weight/weight concentration (% w/w) between 0.1%-10%. If a thinner gel formulation is desired, the thickener is preferably between 1%-5%. If a thicker gel formulation is desired, however, the thickener is preferably between 5%-10%. In some instances, it is preferred if the rheology modifier introduces thixotropic properties to the lubricant composition promotes easier administration as the product thins out during shear stresses induced by administration thereby allowing the composition to flow through restricted cannulas (27 ga-31 ga) more easily that may otherwise not be extrudable. This is an important consideration as RCT moves towards smaller preparations and minimally invasive access openings. Furthermore, thixotropic properties allow the lubricant composition to thin out, becoming more flowable as the endodontic file moves thereby further improving lubricity, debridement, and debris removal. Additional thickeners may be added depending on the consistency of the lubricant composition desired for clinical use. In certain aspects, the lubricant composition may be non-Newtonian or pseudoplastic.

The disclosed lubricant composition may have a viscosity of 1-15,000 mPa·s, of 10-500 mPa·s, or of 1-50 mPa·s. The lubricant composition has a viscosity allowing it to be easily introduced into the root canal for improved penetration to the canal apex, and into dentinal tubules and canal complexities for improved cleansing and debridement. The improved flowability also allows for better debris extraction during instrumentation and helps to clear debris from endodontic file flutes. Preferably, the viscosity of the lubricant composition allows it to be administered into the root canal system through a dental or endodontic tip as small as 31 ga, which is an advantage compared to other products that can only be extruded through much larger 23 ga or 25 ga tips. In certain embodiments, a much thicker lubricant compositions resembles a gel, paste or putty which preferably has a viscosity of 1,000-200,000 Pa·s, 1,500-75,000 Pa·s, and or in some embodiments 2,000-15,000 Pa·s.

Generally, all ingredients in the lubricant composition are hydrophilic or amphiphilic to aid and hasten lubricant removal from the root canal space making the lubricant composition more easily rinsible than currently available products on the market.

Optionally, the lubricant composition includes at least one humectant preferably at a concentration less than or equal to 15%. An example of a suitable humectant is glycerin. Optionally the lubricant composition contains at least one emulsifier at a concentration less than or equal to 5%. An example of a suitable emulsifier is polyoxyethylene sorbitan monooleates of various chain lengths. Optionally, the lubricant composition contains at least one pH modifier to alter the pH to more acidic or basic values depending on the target pH. Optionally, the lubricant composition contains at least one preservative at a concentration less than or equal to 2%. Examples of suitable preservatives include parabens, sorbates, benzoates, amongst others.

Optionally, the lubricant composition contains at least one effervescent agent which could be carbamide peroxide at a concentration less than or equal to 15%. In certain embodiments the effervescent agent is, however, unneeded as surfactants result in sufficient bubbles to aid debris removal. This is another example of a beneficial and unique attribute of the disclosed compositions as the typically used effervescent agent in commercially available products, carbamide peroxide, has limited shelf stability and exhibits unfavorable cytotoxicity. In other words, the disclosure provides lubricant compositions without carbamide peroxide that perform better than existing commercial products that use carbamide peroxide.

Generally, all ingredients should exhibit long-term biocompatibility for safety and toxicity considerations. Furthermore, the selection of ingredients within the lubricant composition may also exhibit more favorable biocompatibility than other lubricant compositions commercially available.

The ratio between composition ingredients may contribute to the observed favorable properties of the lubricant composition.

In further aspects of the disclosure, the lubricant composition provided a suitable shelf life of preferably 6-48 months when stored at room temperature without displaying characteristics of chemical instability or significantly decreased clinical efficacy. The lubricant composition may optionally be stored in a hermetically sealed pouch to minimize moisture loss and to extend shelf life. An example of chemical instability would be an observation of significant phase separation, or the degradation of a chelating agent or antimicrobial agent concentration over time that renders the composition less effective.

In certain aspects, the lubricant composition may be packaged in various syringes, bottles or containers sized between 0.1 mL-4000 mL or syringes sized between 0.5 mL-100 mL. Syringe packaging is preferred as application tips can be mated directly to the syringed of the lubricant composition for immediate application to the root canal.

The packaging of the components must be compatible for long-term storage (months to years). Satisfactory plastic resins for the packaging material may include, but are not limited to, polypropylene, polycarbonate, polyethylene, styrene acrylonitrile, methyl methacrylate-acrylonitrile-butadiene-styrene, poly-cyclohexylenedimethylene terephthalate glycol, among others.

In certain aspects, the lubricant composition is provided as an item within a kit. In some embodiments, the kit may comprise any one or more of the following components: application tips, application brushes, empty syringes, an instructions for use, mixing wells, or other single use vessels, a dental etchant or etchants, a dental cement or cements, an endodontic irrigant or irrigants, an endodontic sealer or sealers, an endodontic file or files, an endodontic reamer or reamers, among other common dental and endodontic products.

The method of using the lubricant composition typically comprises introducing the lubricant composition into the root canal system through the use of various application tips (25 ga-31 ga) attached to a syringe containing the lubricant therein. For thicker viscosities, the lubricant composition may be directly applied to endodontic files, reamers, or other tools, which are then placed in the root canal. In these types of uses, the volume of the lubricant composition may be between 0.1 mL-10 mL but is typically between 0.5 mL and 5 mL. For thinner lubricant compositions, continuous application and flow during instrumentation may be advantageous to help prolong endodontic file longevity and promote debris removal through constant fluid movement. In these types of uses, the volume of the lubricant composition may be between 3 mL and 40 mL but is typically between 3 mL and 15 mL. Generally, the lubricant composition is administered in vivo for seconds to tens of minutes, but rarely exceeding 60 minutes. Usually after the root canal is instrumented using a lubricant, the debris is flushed from the canal and followed by an irrigant regiment (typically sodium hypochlorite and an EDTA based composition) as part of the RCT procedure. The use of the disclosed lubricant composition, however, generally makes a final rinse of an EDTA-based irrigant unnecessary since the lubricant composition removes inorganic matter and debris continuously as it is created during instrumentation. Therefore, the use of the disclosed lubricant composition is advantageous as it simplifies the instrumentation and irrigant procedure/protocol by only utilizing two chemistries (i.e. lubricant composition and NaOCl), which may decrease treatment time and complexity by requiring less steps.

The formulation of the lubricant composition allows a dental professional to more easily prepare the root canal space during the instrumentation step of the RCT procedure. By more effectively removing debris during the instrumentation step, while also keeping the endodontic file flutes clear and the endodontic file edges lubricated, the lubricant composition allows a dental professional to fully prepare a root canal to working length using only two to three endodontic files compared to four to six using conventional techniques. Not only does this save the dental professional the cost of additional endodontic files, but this saves considerable procedural time by effectively instrumenting the canal in less than half the time compared to conventional techniques.

Although specific details and examples are provided for the lubricant composition's use during RCT instrumentation, the composition may be useful during RCT irrigation as well as many oral and dental applications. For example, the lubricant composition may be useful during vital pulp therapy, cavity disinfection, pediatric pulpotomy, revascularization procedures, restorative preparations when treating carious lesions, periodontal disease treatment, implant installation, amongst others. Furthermore, the lubricant compositions may be useful for cleansing and debriding hard surfaces (e.g., dental equipment, countertops).

The method of manufacturing an example lubricant composition with a neutral to slightly basic pH is as follows: first a portion of the water is placed into a mixing vessel and the antimicrobial agent (e.g., chlorhexidine) is added; this forms “mixture 1”. The remainder of the water is added in a separate mixing vessel. Then the pH modifier is added and mixed until dissolved such that the mixture is around a target neutral to slightly basic pH value, followed by addition of the chelating agent (e.g., EDTA); this forms “mixture 2”. The remaining surfactants must be added after the chelating agent is fully dissolved in mixture 2. Next, the surfactants are added to mixture 2 in the following order: ethoxylated alcohol (e.g., 2-propenoic acid) and betaines (e.g., lauramidopropyl betaine and myristamidopropyl betaine). Mixture 1 is then added to mixture 2 and allowed to fully mix creating “mixture 3”. Finally, the fluorosurfactant (e.g., Masurf FS-2825 or Thetawet FS-8150) is dissolved in mixture 3. The order of addition for the surfactants is critical to the combination of the formula; if ingredients are combined out of order, the solution will not be clear in appearance and may not be sufficiently effective. Additionally, for certain antimicrobial agents, it is necessary to add at a specific mixture step or pH such that precipitation of the antimicrobial agent does not occur. If thicker consistencies are desired, then the rheology modifier may be added to the liquid mixture 3 at the end to create a gel-like consistency. In certain embodiments, it is advantageous to manufacture the lubricant composition in an asymmetric centrifuge mixer. In certain embodiments, the chelating agent may be kept in a stock aqueous solution (e.g., 5% to 20% EDTA stock solution) with at least one pH modifier, at least one antimicrobial agent and at least one surfactant for ease of manufacturing.

The method of manufacturing an example lubricant composition with a neutral to acidic pH is as follows: first the water is placed into a mixing vessel and the chelating agents (e.g., citric acid) are dissolved. The mixture is brought to a target neutral to acidic pH using a pH modifier (e.g., hydrochloric acid). An antimicrobial agent (e.g., elemental iodine, I₂) is next added to the mixture. In certain embodiments, the antimicrobial agent may be kept in a stock aqueous solution (e.g., 2% to 5% I₂ stock solution) for case of manufacturing. Next, the lubricant and optional humectant are added to the mixture followed by the surfactants (e.g., ethoxylated alcohol).

### Definitions

For purposes of interpreting this specification, the following abbreviations, terms and definitions will apply and whenever appropriate, terms used in the singular will also include the plural and vice versa. In the event that any definition set forth below conflicts with any document incorporated herein by reference, the definition set forth below shall control.

The term “room temperature” or ambient temperature as used herein refers to common ambient temperatures ranging from about 18° C. to about 27° C.

The term “treating” refers to administering a therapy in an amount, manner, or mode effective to improve a condition, symptom, or parameter associated with a disorder. In some aspects, treating refers to the treatment of a dental ailment such as an infected tooth.

The term “substantially” as used herein means to a great or significant extent, but not completely.

As used herein, “a”, “an”, “the”, “at least one”, and “one or more” are used interchangeably.

The terms “comprises” and variations thereof do not have a limiting meaning where these terms appear in the description and claims.

The term “patient” or “subject” refers to mammals and humans. Thus, in one aspect, the subject is a mammal, or a mammal in need thereof. In one aspect, the subject is a human, or human in need thereof. In one aspect, the human or human in need thereof is a medical patient. The subject can be from ˜0 years of age to 99 years of age or older.

The term “in vivo” generally means in a living subject.

The term “composition” generally refers to the chemical makeup of certain embodiments of the disclosed invention and is synonymous with “formula”.

The term “chemical stability” generally refers to a composition or formula that remains in chemical equilibrium for a period of time without significant reactivity. In some instances, this stability can be observed visually, for example, if there is not a change in the composition's state, such as observing the formation of a visible solid precipitate over time or phase separation. Said differently, the observation of a visible precipitate or phase separation within a composition or formula over time would indicate the initial formula was chemically instable. The precipitate then formed due to the composition desiring to became more chemically and thermodynamically stable.

The term “endodontic” generally refers to inside the tooth. The term “endodontic procedure” is generally synonymous with “root canal therapy”, or “root canal procedure”, and refers to a treatment of an infected tooth to cleanse the root canal system and remove the infection.

The term “smear layer” is known to those of skill in the art of dentistry and refers to the complex accumulation of soluble and insoluble organic and inorganic debris resulting from the mechanical preparation of a tooth surface. The smear layer includes cutting debris, tooth particles, microorganisms, necrotic material, and other substances resulting from preparation, and can include a superficial layer on the surface of a prepared tooth along with a layer or layers that are packed into the adjacent dentinal tubules at varying depths.

The term “root canal system” generally refers to the naturally occurring anatomical spaces within the root(s) of a tooth including the pulp chamber.

The term “gel” generally refers to the material consistency, where a thickener, or rheology modifier, has been added, such that the material is appreciably thicker than a liquid form. A gel may provide the ability of a material to better cling to surrounding surfaces. The term “gel” may be understood to be synonymous to “cream”, “lotion”, “paste”, “balm”, “ointment”, “salve”, “grease”, and the like.

The term “chelating agent” generally refers to chemical compounds or molecules that complex with metal ions to form a stable, water-soluble complex. The term “chelating agent” is to be understood as synonymous with the terms “chelant,” “chelator,” “metal complexing agent,” “complexing agent,” “sequestrant,” and “sequestering agent.”

The abbreviation “EDTA” will be understood to refer to the chemical ethylenediaminetetraacetic acid, which is a chelating agent capable of binding a variety of metal ions, for example, calcium ions.

The abbreviation “HEDP” will be understood to refer to the chemical 1-hydroxyethylidene-1,1-diphosphonic acid, which is a chelating agent capable of binding a variety of metal ions, for example, calcium ions.

The abbreviation “PBTC” will be understood to refer to the chemical 2-phosphonobutane-1,2,4-tricarboxylic acid, which is a chelating agent capable of binding a variety of metal ions, for example, calcium ions.

The term “dentin” generally refers to a type of calcified tissue that is a major component of teeth. Hydroxyapatite is the main inorganic constituent of dentin and is a naturally occurring mineral from of calcium apatite with the formula Ca₅(PO₄)₃(OH).

The term “lubricant composition” generally refers to embodiments of the present disclosure that are generally used as lubricants and debriding materials during RCT, and most specifically during the instrumentation portion of RCT (although the disclosed compositions may be used at any point in the RCT procedure as well as other dental and oral applications).

The term “working length” generally refers to the distance from a coronal reference point to the point at which canal preparation and obturation should terminate.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### EXAMPLES

Table 1 summarizes the chemical constituents that comprise the disclosed lubricant composition. Additionally, Table 2 provides example formulas compositions of the disclosure exhibiting various gel-like thicker consistencies. Lastly, Table 3 provides example formulas compositions of the disclosure exhibiting water-like consistencies.

TABLE 1
Chemical constituents that comprise
the disclosed lubricant composition.

| | | | | More |
|---|---|---|---|---|
| | | | Preferred | Preferred |
| | | | Concen- | Concen- |
| | | | tration | tration |
| Ingredient | Example | Required | (w/w %) | (w/w %) |
| Solvent | Distilled Water | Yes | 20-90% | 40-80% |
| Chelating agent | EDTA | Yes | 0.5-40% | 5-20% |
| Lubricant | Propylene Glycol | Yes | 0.5-15% | 2.5-10% |
| Surfactant | Tomadol 91.6 | Yes | 0.5-5% | 1-2% |
| Rheology | Fumed Silica | Optional | 0-10% | 0-6% |
| Modifier | | | | |
| Thickener & | Polyoxyethylene | Optional | 0-5% | 0.5-1% |
| Emulsifier | (80) sorbitan | | | |
| | monooleate | | | |
| Humectant | Glycerin | Optional | 0-15% | 2.5-10% |
| pH Modifier | 50% NaOH | Optional | 0-10% | 2-5% |
| Antimicrobial | Chlorhexidine | Optional | 0-5% | 0-2% |
| agent | | | | |

TABLE 2
Example formulas or compositions that comprise the disclosed lubricant
composition exhibiting thicker gel-like consistencies.

| | | For- | For- | For- |
|---|---|---|---|---|
| | Classifi- | mula A | mula B | mula C |
| Ingredient | cation | (w/w %) | (w/w %) | (w/w %) |
| EDTA | Active | 10.40% | 10.40% | 10.40% |
| Fumed Silica | Rheology | 2% | 4% | 6% |
| | Modifier | | | |
| Polyoxyethylene | Thickener | 0.7% | 0.7% | 0.7% |
| (80) sorbitan | | | | |
| monooleate | | | | |
| Glycerin | Thickener/ | 5% | 5% | 5% |
| | Humectant | | | |
| Propylene Glycol | Lubricant | 5% | 5% | 5% |
| Tomadol 91.6 | Surfactant | 1.14% | 1.14% | 1.14% |
| Mackam LMB-K | Surfactant | 1.20% | 1.20% | 1.20% |
| Thetawet FS-8150 | Surfactant | 0.02% | 0.02% | 0.02% |
| Chlorhexidine | Antimicro- | 0.3% | 0.3% | 0.3% |
| | bial Agent | | | |
| Acusol 445 | Surfactant | 2.72% | 2.72% | 2.72% |
| 50% NaOH | pH Modifier | 3.07% | 3.07% | 3.07% |
| Distilled Water | Solvent | 68.44% | 66.44% | 64.44% |

| | | | |
|---|---|---|---|
| TOTAL | 100% | 100% | 100% |
| pH | 7-7.5 | 7-7.5 | 7-7.5 |
| Consistency | Thinnest gel | Thin gel | Thick gel |

TABLE 3
Example formulas or compositions that comprise the disclosed
lubricant composition exhibiting a water-like consistency.

| | | For- | For- | For- |
|---|---|---|---|---|
| | Classifi- | mula D | mula E | mula F |
| Ingredient | cation | (w/w %) | (w/w %) | (w/w %) |
| Citric acid | Chelator | 13.00% | 13.00% | 13.00% |
| monohydrate | | | | |
| Polyacrylic acid | Chelator | 2.50% | 2.50% | 2.50% |
| NaOH (50%) | pH Modifier | 0.68% | 2.52% | 4.35% |
| Propylene Glycol | Lubricant | 5.00% | 5.00% | 5.00% |
| Glycerin | Thickener/ | 5.00% | 5.00% | 5.00% |
| | Humectant | | | |
| Dowfax C6L | Surfactant | 2.50% | 2.50% | 2.50% |
| Tomadol 91-6 | Surfactant | 1.00% | 1.00% | 1.00% |
| Elemental Iodine (I2) | Antimicro- | 0.15% | 0.15% | 0.15% |
| | bial Agent | | | |
| Distilled Water | Solvent | 70.17% | 68.33% | 66.50% |

| | | | |
|---|---|---|---|
| TOTAL | 100% | 100% | 100% |
| pH | 2-2.5 | 3-3.5 | 4-4.5 |
| Consistency | Water-like | Water-like | Water-like |

To illustrate improved extrudability of the lubricant compositions, 12 mL syringes were filled with RC Prep (Premier Dental Products, Plymouth Meeting, PA, USA) and the disclosed Formulas A-F. Dental application tips (23 ga, 25 ga, 27 ga, and 30 ga; Appli-Vac Tips, Vista Apex, Racine, WI, USA) were mated to the syringes and material was attempted to be hand extruded through the tips as would be performed during a clinical procedure. RC Prep was only able to be extruded through 23 ga and 25 ga tips, albeit with a lot of force and hand fatigue, while Formulas A-F were able to be extruded through all tips with ease (even Formula C which is the thickest gel). The thixotropic nature of the lubricant composition gels (Formulas A-C) allows for extrusion through these small orifice application tips. Therefore, in certain embodiments, the lubricant composition can be administered into the root canal system through a dental or endodontic tip as small as 31 ga, which is an advantage compared to other products that can only be extruded through much larger 23 ga or 25 ga tips.

To illustrate improved biocompatibility of the disclosed lubricant composition, a MEM elution cytotoxicity test was performed by Geneva Labs (Elkhorn, WI, USA) following ANSI/AAMI/ISO 10993-5/(R)2014 and USP 87 standards. Test groups included Formula C, Formula D and RC Prep (Premier Dental Products, Plymouth Meeting, PA, USA), along with controls. Briefly, each material was incubated at various dilutions (neat, 1:2, 1:5, 1:10, and 1:50) with L-929 cells (ATCC CCL-1) for 24 and 72 hours. Following incubation, cell monolayers were examined microscopically for cell morphology and lysing, and a grade of 0-4 was provided. Each material dilution was tested in triplicate. Results are shown in Table 4 below. Inconclusive results were observed at various timepoints and dilutions for Formula C & Formula D (due to a precipitate being observed) although non-cytotoxic results were obtained for other timepoints and dilutions. Comparatively, RC Prep was observed to be severely cytotoxic at all timepoint and dilutions. Therefore, the disclosed lubricant compositions exhibit more favorable biocompatibility than products commercially available.

TABLE 4
MEM elution cytotoxicity testing of Formula C versus RC Prep.

| Device Name | Concentration | Score (AVE ± SD) | Result |
|---|---|---|---|
| Formula C | Neat | Inconclusive | Inconclusive |
| (24 hours) | 1:2 | Inconclusive | Inconclusive |
| | 1:5 | Inconclusive | Inconclusive |
| | 1:10 | 0 ± 0 | Non-cytotoxic |
| | 1:50 | 0 ± 0 | Non-cytotoxic |
| Formula C | Neat | Inconclusive | Inconclusive |
| (72 hours) | 1:2 | Inconclusive | Inconclusive |
| | 1:5 | 1 ± 0 | Non-cytotoxic |
| | 1:10 | 1 ± 0 | Non-cytotoxic |
| | 1:50 | 0 ± 0 | Non-cytotoxic |
| Formula D | Neat | Inconclusive | Inconclusive |
| (24 hours) | 1:2 | 4 ± 0 | Cytotoxic |
| | 1:5 | 4 ± 0 | Cytotoxic |
| | 1:10 | 1 ± 0 | Non-cytotoxic |
| | 1:50 | 0 ± 0 | Non-cytotoxic |
| Formula D | Neat | 4 ± 0 | Cytotoxic |
| (72 hours) | 1:2 | 4 ± 0 | Cytotoxic |
| | 1:5 | 4 ± 0 | Cytotoxic |
| | 1:10 | 1 ± 0 | Non-cytotoxic |
| | 1:50 | 0 ± 0 | Non-cytotoxic |
| RC-Prep | Neat | 4 ± 0 | Cytotoxic |
| (24 hours) | 1:2 | 4 ± 0 | Cytotoxic |
| | 1:5 | 4 ± 0 | Cytotoxic |
| | 1:10 | 4 ± 0 | Cytotoxic |
| | 1:50 | 4 ± 0 | Cytotoxic |
| RC-Prep | Neat | 4 ± 0 | Cytotoxic |
| (72 hours) | 1:2 | 4 ± 0 | Cytotoxic |
| | 1:5 | 4 ± 0 | Cytotoxic |
| | 1:10 | 4 ± 0 | Cytotoxic |
| | 1:50 | 4 ± 0 | Cytotoxic |

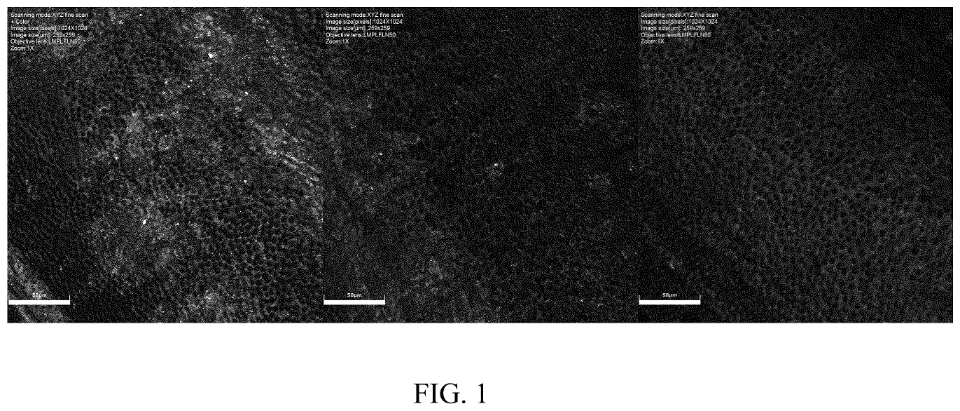

To demonstrate satisfactory smear layer removal using the disclosed lubricant composition, an ex vivo study was performed on extracted single rooted human teeth. Briefly, teeth were decoronated to a standard 18 mm length. Working length was established using a K15 hand file. The disclosed lubricant composition (Formula D) was continuously administered within the coronal chamber while the instrumentation process ensued using three endodontic files (15/02, 25/02 and 35/04). Approximately 6 mL of Formula D was used during the instrumentation procedure, which was followed by administration of 6 mL of 6% NaOCl using a 30 ga side vent irrigation tip. Teeth were sectioned longitudinally and imaged using a laser microscope (Olympus LEXT OLS400). Representative results are shown in FIG. 1 which consists of laser microscope images that show satisfactory smear layer removal within the root canal using the disclosed lubricant composition. The left image in FIG. 1 shows the coronal third of the canal, the middle image shows the middle third of the canal, and the right image shows the apical third of the canal.

What is claimed is:

1. An antimicrobial endodontic lubricant, comprising:
at least one chelating agent;
at least one lubricant;
at least one surfactant,
at least one debriding agent,
at least one antimicrobial agent; and,
at least one solvent
wherein, the at least one debriding agent and at least one antimicrobial agent are the same chemical; and,
wherein, the composition exhibits a pH between 2.0 and 4.5.

2. The composition of claim 1, additionally comprising at least one humectant.

3. The composition of claim 1, additionally comprising at least one debriding agent.

4. The composition of claim 1, additionally comprising at least one rheology modifier.

5. The composition of claim 1, additionally comprising at least one stabilizer.

6. The composition of claim 1, wherein at least one chelating agent is citric acid.

7. The composition of claim 1, wherein at least one chelating agent is EDTA.

8. The composition of claim 1, wherein at least one chelating agent is polyacrylic acid.

9. The composition of claim 1, wherein the at least one chelating agent consists of citric acid and polyacrylic acid.

10. The composition of claim 1, wherein the at least one chelating agent consists of citric acid and polyacrylic acid, wherein the polyacrylic acid has a molecular weight between 1000 g/mol and 10000 g/mol.

11. The composition of claim 1, wherein the antimicrobial agent is elemental iodine (I₂).

12. The composition of claim 1, wherein the antimicrobial agent is elemental iodine (I₂) at a concentration between 300 PPM and 3000 PPM.

13. The composition of claim 1, additionally comprising a pH modifier.

14. The composition of claim 1, wherein the at least one lubricant is propylene glycol, glycerin or a combination thereof.

15. A method of treating a tooth, comprising:
introducing an antimicrobial endodontic lubricant into a root canal or root canals to lubricate at least one endodontic file,
wherein, the antimicrobial endodontic lubricant inhibits the buildup of dentinal debris within the flutes of the at least one endodontic file; and,
wherein, the antimicrobial endodontic lubricant is comprised of:
at least one chelating agent;
at least one lubricant;
at least one surfactant,
at least one debriding agent,
at least one solvent, and;
wherein, the composition exhibits a pH between 2.0 and 4.5.

16. The method of claim 15, wherein the antimicrobial endodontic lubricant composition additionally comprises at least one antimicrobial agent.

17. The method of claim 15, wherein the antimicrobial endodontic lubricant composition additionally comprises at least one antimicrobial agent that is the same chemical of compound as the at least one debriding agent.

18. The method of claim 15, wherein the antimicrobial endodontic lubricant composition additionally comprises at least one antimicrobial agent, wherein the antimicrobial agent is elemental iodine (I₂) at a concentration between 300 PPM and 3000 PPM.

19. The method of claim 15, wherein the at least one chelating agent consists of citric acid and polyacrylic acid.

20. The method of claim 15, wherein the at least one lubricant is propylene glycol, glycerin or a combination of the two.
